(19) United States
(12) Patent Application Publication
(10) Pub. No.: US 2025/0354113 A1
(43) Pub. Date: Nov. 20, 2025

(54) INNER EAR ORGANOIDS HAVING IMPROVED HAIR CELL DIFFERENTIATION ABILITY AND METHOD FOR PRODUCING SAME

(71) Applicant: AJOU UNIVERSITY INDUSTRY-ACADEMIC COOPERATION FOUNDATION, Suwon-si (KR)

(72) Inventors: Yun-Hoon Choung, Seoul (KR); Yeon Ju Kim, Suwon-si (KR)

(21) Appl. No.: 18/701,144

(22) PCT Filed: Oct. 14, 2022
(86) PCT No.: PCT/KR2022/015663

§ 371 Date: Apr. 12, 2024

(30) Foreign Application Priority Data
Oct. 15, 2021 (KR) 10-2021-0137546

(51) Int. Cl. C12N 5/071

(52) U.S. Cl. CPC C12N 5/0625; C12N 2501/415; C12N 2501/999; C12N 2513/00

(57) ABSTRACT

The present invention relates to a method for producing inner ear organoids having improved hair cell differentiation ability, and the inner ear organoids produced according to the same. The present invention is based on the discovery that treatment with a gamma secretase inhibitor and a Wnt signaling activator during the production of inner ear organoids can promote the differentiation of hair cells, and provides the timing of the treatment with a gamma secretase inhibitor and a Wnt signaling activator at which the amount of hair cells in the inner ear organoids can be maximized. The present technology may be used as a method for increasing the production efficiency of inner ear organoids to be applied in the field of hearing loss treatment.

### CROSS-REFERENCE TO RELATED APPLICATION

This application claims priority to and the benefit of Korean Patent Application No. 10-2021-0137546, filed on Oct. 15, 2021, the disclosure of which is incorporated herein by reference in its entirety.

### TECHNICAL FIELD

The present invention relates to an inner ear organoid and a method for producing the same, and more specifically provides a method for producing an inner ear organoid having improved hair cell differentiation ability by specifying the treatment timing of a gamma secretase inhibitor and a Wnt signaling activator in the inner ear organoid formation process, and an inner ear organoid including a large amount of hair cells.

### BACKGROUND ART

Hearing loss refers to a phenomenon in which a person cannot hear well due to abnormalities in the outer, middle or inner ear. Most of the occurrence of hearing loss can be classified into aging, sudden, noise, ototoxicity, hereditary and the like, and it is mainly characterized by the damage and death of auditory hair cells in the Organ of Corti of the inner ear.

In the hearing loss research, it is difficult to obtain cells derived from actual patients, and animal auditory cell lines and cochlear tissue cannot mimic the actual inner ear, which limits the development of drugs for preventing and treating hearing loss. Additionally, in a situation where human and laboratory animal research is increasingly limited due to ethical issues, organoids are a groundbreaking technique in that they reproduce real organs by using human and animal stem cells.

An organoid is a three-dimensional ‘organoid’ that similarly reproduces a body organ used as a research model in vitro, which enables high-speed drug screening based on organoid mass production technology, and it has the great advantage of enabling disease modeling and customized therapeutic agent screening with genetic information from actual patient tissues.

A three-dimensional culture protocol has been developed for inner ear organoids since 2014, but since the organoid production efficiency is very low and the number of hair cells in the produced organoids is very small at about 1% of the total cells, there is a problem in that it is not suitable for screening medicaments.

Auditory hair cells in the Organ of Corti, which are auditory receptors inside the cochlea, are first expressed in the sensory epithelium before support cells, and differentiation between hair cells and support cells is determined through the lateral inhibition of Notch signals. Gamma secretase (t-secretase) is a hydrolytic enzyme that cleaves the transmembrane site of Notch, and it liberates the Notch intracellular domain (NICD) into cells. In addition, when NICD moves into the nucleus, Atoh1, which is a transcription factor associated with hair cell differentiation, is suppressed, and genes such as Hes1 and Hes5 are expressed. Therefore, gamma secretase inhibitors can induce hair cells from support cells by reducing the expression level of NICD.

Wnt signaling regulates various developmental processes such as organ development, cell proliferation and cell fate determination. Wnt signaling is known to be important for sensory progenitor cell proliferation and hair cell differentiation in the embryonic stage by stabilizing β-catenin due to the inhibition of GSK-3, activity, which enters the nucleus and acts as a Tcf/Lef transcription factor to activate target genes.

Therefore, the inventors of the present invention completed the present invention by confirming that in order to maximize hair cell differentiation in an organoid production process similar to the differentiation process from non-sensory cells to hair cells in vivo, the inhibition of Notch signaling through a gamma secretase inhibitor and Wnt signaling activator efficiently increases hair cells in inner ear organoids.

### DISCLOSURE

### Technical Problem

The present invention is directed to providing an inner ear organoid containing a large amount of hair cells with improved differentiation ability of hair cells and a method for producing the same.

In addition, the present invention is directed to providing a gamma secretase inhibitor and a Wnt signaling activator as compounds that are capable of maximizing the differentiation of hair cells when producing an inner ear organoid, and providing treatment timing thereof.

However, the technical problems to be achieved by the present invention are not limited to the above-mentioned problems, and other problems that are not mentioned will be clearly understood by those skilled in the art from the description below.

### Technical Solution

Hereinafter, the present invention will be described in detail. The advantages and features of the present invention and the exemplary embodiments described below will become clear with reference to the exemplary embodiments that achieve the same. However, the present invention is not limited to the exemplary embodiments disclosed below, but may be implemented in various different forms, and the present exemplary embodiments are only provided to complete the disclosure of the present invention and to fully inform the scope of the invention to those skilled in the art to which the present invention pertains, and the present invention is only defined by the scope of the claims. Like reference numerals designate like elements throughout the specification.

Unless otherwise defined, all terms (including technical and scientific terms) used in the present specification may be used in meanings that are commonly understood by those of ordinary skill in the art to which the present invention pertains. In addition, terms defined in commonly used dictionaries are not interpreted ideally or excessively unless explicitly specifically defined. Terminology used in the present specification is for describing the exemplary embodiments and is not intended to limit the present invention. In the present specification, singular forms also include plural forms unless specifically stated otherwise in a phrase.

The present invention provides a method for producing an inner ear organoid having improved hair cell differentiation ability, including the steps of a) culturing embryonic stem cells or induced pluripotent stem cells to induce the formation of endoderm, ectoderm and otic vesicle of the cultured cells; and (b) maturing by culturing the cells differentiated according to step (a) in organoid medium, wherein a gamma secretase inhibitor and Wnt signaling activator are treated in step (b).

As used herein, the term “organoid” is also referred to as an organoid, and it refers to a three-dimensional cell aggregate body formed through self-renewal and self-organization from adult stem cells (ASC), embryonic stem cells and induced pluripotent stem cells (iPSC). Organoids are ex vivo three-dimensional organs that have a small, simplified form that mimics the anatomy of real tissues. By constructing an organoid from patients' tissue, it enables disease modeling and drug screening through repeated tests based on the patient's genetic information.

As used herein, “hair cell” means a cell located in the Organ of Corti of the cochlea in the inner ear, and “differentiation” refers to the ability of cells to specialize in structure and function while dividing, proliferating and growing. The inner ear organoid prepared according to the present invention may contain a large amount of hair cells, because the differentiation ability of hair cells is improved.

As used herein, the term “stem cell” refers to a cell having the ability to differentiate into various cells and the ability to self-proliferate through suitable environments and stimuli, and it may be an adult stem cell, an induced pluripotent stem cells or an embryonic stem cell, and preferably, it may be an embryonic stem cell or an induced pluripotent stem cells.

As used herein, the term “differentiation” refers to a phenomenon in which daughter cells produced by cell division acquire functions different from those of the original parent cell, and refers to a process in which functions specific to a specific tissue or cell are acquired, and “differentiation” in the present invention may mean the “differentiation” of cultured embryonic stem cells or induced pluripotent stem cells. In the present invention, the “start of differentiation” may refer to a step in which the formation of endoderm, ectoderm or otic vesicle of cultured cells begins, and preferably, it may mean a step in which the formation of true endoderm in cultured cells begins.

The process of forming an inner ear organoid basically includes the steps of (1) the formation of a definitive ectoderm, (2) the formation of a non-neural ectoderm, (3) the preplacodal ectoderm, (4) the formation of an otic-epibranchial placode domain, (5) the formation of an otic vesicle, and (6) the differentiation of hair cells, support cells and neurons in order.

In the present specification, the “maturation step” may mean a step after the otic vesicle formation step, and it may be divided into a first maturation step of culturing cells in N₂ medium and a second maturation step of culturing cells in organoid medium. The first maturation step may include the formation of otic vesicles.

According to an exemplary embodiment of the present invention, the gamma secretase inhibitor and the Wnt signaling activator may be treated in the second maturation step of culturing cells in an organoid medium, more preferably, it may be treated in the later stage of the second maturation step.

According to an exemplary embodiment of the present invention, considering that it takes about 30 days for mESCs and about 90 days for hiPSCs to form an organoid, the gamma secretase inhibitor and the Wnt signaling activator may be treated for about 5 days to 7 days after day 25 (day 25 to day 35) after the start of differentiation of mESCs, and after day 80 (about day 80 to day 90) after the start of differentiation of hiPSCs. Most preferably, the gamma secretase inhibitor and the Wnt signaling activator are treated for a period of day 28 to day 34 day after the start of differentiation for mESCs, and for a period of day 80 to day 86 after the start of differentiation for hiPSCs.

As used herein, the term “gamma secretase (γ-secretase) inhibitor” may include N—[N-(3,5-Difluorophenacetyl)-L-alanyl]-S-phenylglycine t-butyl ester (DAPT), (5S)-(tert-butoxycarbonylamino)-6-phenyl-(4R)-hydroxy-(2R)-benzylhexanoyl)-L-leu-L-phe-amide (L-685,458) and (S,S)-2-[2-(3,5-difluorophenyl)-acetylamino]-N-(1-methyl-2-oxo-5-phenyl-2,3-dihydro-1H-benzo[e][1,4]diazepin-3-yl)-propionamide (compound E), but the present invention is not limited thereto, and most preferably, it is DAPT.

As the “Wnt signaling activator” of the present invention, a Wnt activator commonly used in the art may be used, and preferably, CHIR99021 may be used.

According to an exemplary embodiment of the present invention, the Wnt signaling activator may be treated at a concentration of 1 μM to 2.5 μM (preferably 2.5 μM), and the gamma secretase inhibitor may be treated at a concentration of 1 to 5 μM (preferably 5 μM) in the later stage of the organoid maturation step.

As used herein, the term “medium” refers to a medium that is capable of supporting the proliferation, survival and differentiation of inner ear organoids in vitro, and it includes all conventional media that are suitable for culturing and differentiating inner ear organoids used in the field. Depending on the type of cell, the type of medium and culture conditions may be appropriately selected.

In addition, the present invention provides an inner ear organoid having improved hair cell differentiation ability, which is produced by the organoid product method.

### Advantageous Effects

The method for producing an organoid having improved hair cell differentiation ability according to the present invention can increase the efficiency of producing an inner ear organoid, and can provide an organoid culture method for drug screening.

Since the organoid produced according to the present invention has an increased amount of hair cells than inner ear organoids according to a general production method, it has an efficient advantage in the hearing loss disease modeling and screening.

### DESCRIPTION OF DRAWINGS

FIG. 1 is a mimetic diagram of a known method for producing an inner ear organoid, a of FIG. 1 shows the time-sequential process of inner ear organoid formation in vivo and in vitro of ESCs, and b of FIG. 1 is a mimetic diagram comparing the steps of inner ear organoid production of mESC (mouse embryonic stem cell) and hiPSC (human induced pluripotent stem cell) in chronological order.

FIG. 2 is a mimetic diagram showing the method for producing an inner ear organoid having improved hair cell differentiation efficiency according to an exemplary embodiment of the present invention. The upper part of FIG. 2 is an image showing the differentiation process of cells for each production step.

FIG. 3 is a set of images showing the cell shape of the mESC cell aggregate according to the production method and inner ear organoid culture method of the present invention in chronological order.

FIG. 4 is a set of images confirming whether sensory hair cells are generated through Myosin VIIA, Brn3c and Sox2 immunostaining of the inner ear organoid produced according to an exemplary embodiment of the present invention, and the bottom right is an image confirming the formation of cilia in the inner ear organoid produced according to an exemplary embodiment of the present invention through transmission electron microscopy.

FIG. 5 is the results of comparing the characteristics of the organoids when a gamma secretase inhibitor and a Wnt activator were treated in the early stage of the inner ear organoid maturation step according to an exemplary embodiment of the present invention, wherein A is a set of images comparing morphological changes, B is a graph analyzing the size of organoids and the amount of buds produced, C is the results of immunostaining for inner ear hair cell (MyoVIIA) and feeder cell (Sox2) markers, and D is the qRT-PCR results for MyoVIIA.

### MODES OF THE INVENTION

Hereinafter, the examples will be described in detail to aid understanding of the present invention. However, the following examples are merely illustrative of the content of the present invention, but the scope of the present invention is not limited to the following examples. The examples of the present invention are provided to more completely explain the present invention to those skilled in the art.

### Example 1

### 1-1. Method for Producing Inner Ear Organoid

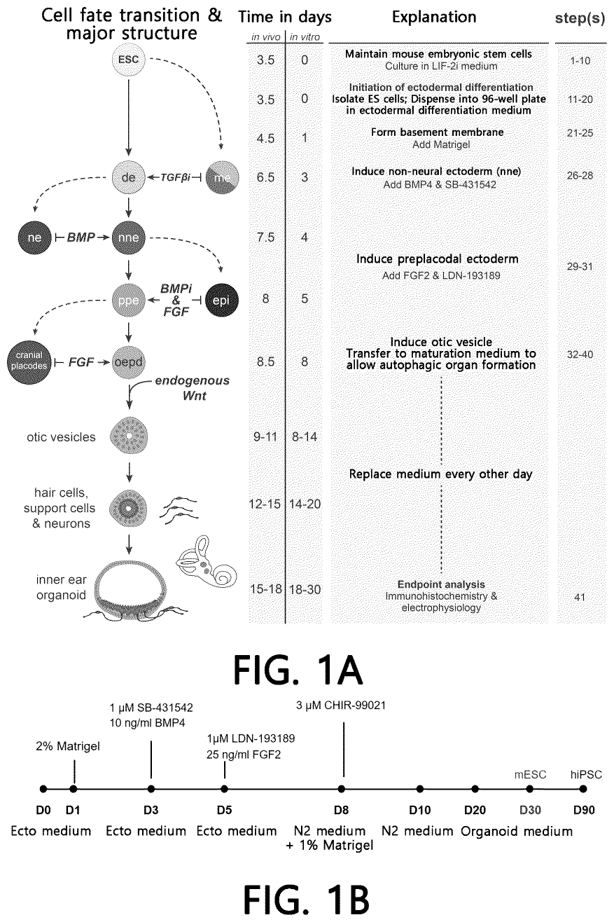

ESCs were differentiated into an inner ear-like organoid based on prior literature [Koehler K R, Hashino E. 3d mouse embryonic stem cell culture for generating inner ear organoids. Nature Protocols 2014; 9: 1229-44]. Briefly, on day 3 of differentiation, non-neural ectoderm was induced by adding 10 ng/mL of recombinant BMP4 (Stemgent, Beltsville, MD, USA) and 1 μM SB431542 (Stemgent) to the cultured cells (non-neural ectoderm induction step). On day 5, preplacodal ectoderm was induced by adding 25 ng/mL of FGF-2 (Peprotech, Rocky Hill, NJ, USA) and 1 μM of LDN-193189 (Stemgent) (preplacodal ectoderm induction step). Cells were cultured for 2 days, and the medium was replaced with maturation medium containing 1% Matrigel on day 8 (otic vesicle induction and first maturation step). Half of the medium was replaced with Matrigel-free maturation medium every other day until day 18, and then, the organoid was cultured in the organoid medium (second maturation step). The schematic diagram of the differentiation process is shown in FIG. 1.

Organoids in the following examples were produced according to the above method unless otherwise specified.

### 1-2. Fabrication of Inner Ear Organoid Having Improved Hair Cell Differentiation Ability

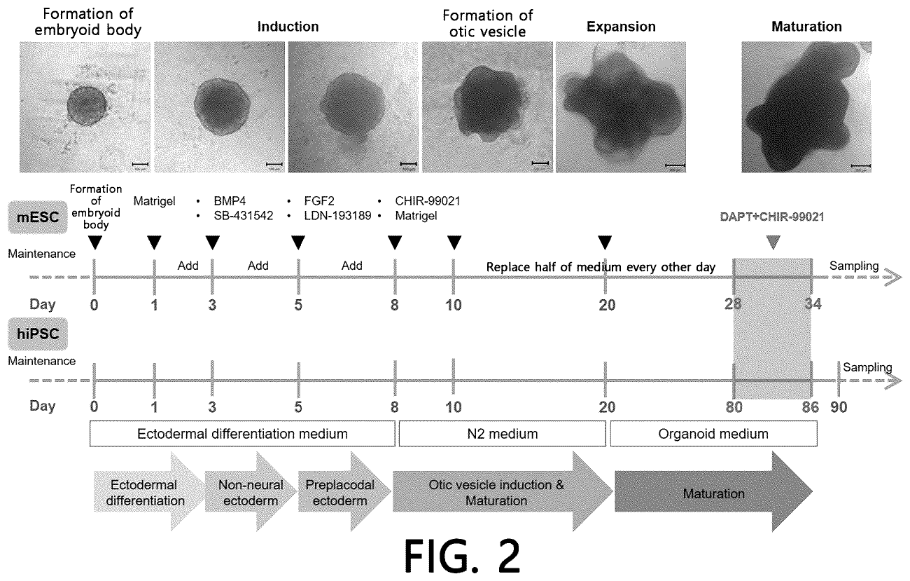

Among the organoid production steps according to Example 1-1, the second maturation stage of organoids was set at 20 to 34 days after differentiation, and during the second maturation step, for 6 days from day 28, cells were treated with a mixture of 5 μM of DAPT, which is a gamma secretase inhibitor, and 2 μM of CHIR-99021, which is a Wnt signaling activator. (refer to FIG. 2).

### Example 2

### Treatment of DAPT and CHIR-99021 in Early Stage of Organoid Maturation Step

In order to compare the degree of differentiation of hair cells according to the treatment period of DAPT and CHIR-99021 during the organoid production steps, DAPT and CHIR-99021 were treated in the early stage of maturation in this example.

Among the organoid production steps according to Example 1-1, the second maturation stage of organoids was set at 20 to 34 days after differentiation, and 5 μM of DAPT, which is a gamma secretase inhibitor, and 2 μM of CHIR-99021, which is a Wnt signaling activator, were treated for 6 days starting from day 8 after differentiation.

### Example 3 Analysis of Hair Cell Efficiency in Inner Ear Organoid Using Gamma Secretase Inhibitor and Wnt Signal Activator

In the organoids produced according to Example 1 and Example 2, and in a control group treated with DMSO instead of the gamma secretase inhibitor and Wnt signaling activator in the maturation stage, the number of the occurrence of organoid buds that differentiated into the inner ear was analyzed, and the incidence of hair cells was confirmed by using myosin7a antibody.

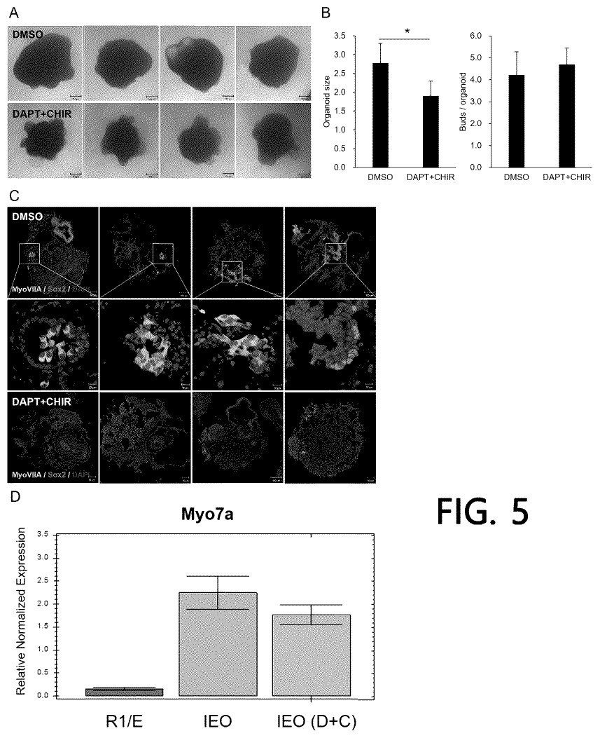

The organoid produced according to Example 2 showed a significant decrease in organoid size compared to the DMSO control group (FIGS. 5 A and B), and it can be confirmed through immunostaining and quantitative RT-PCR that the number of hair cells was significantly reduced (FIGS. 5 C and D).

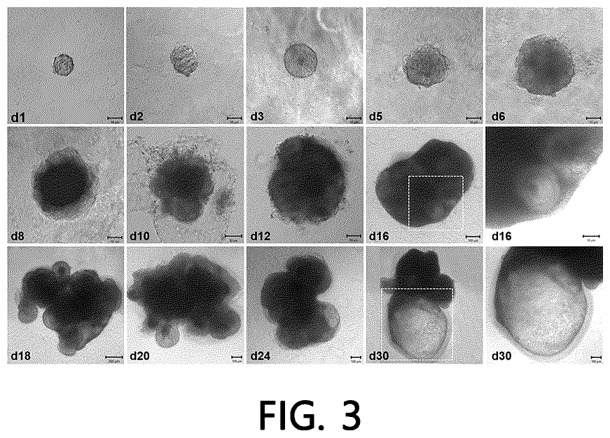

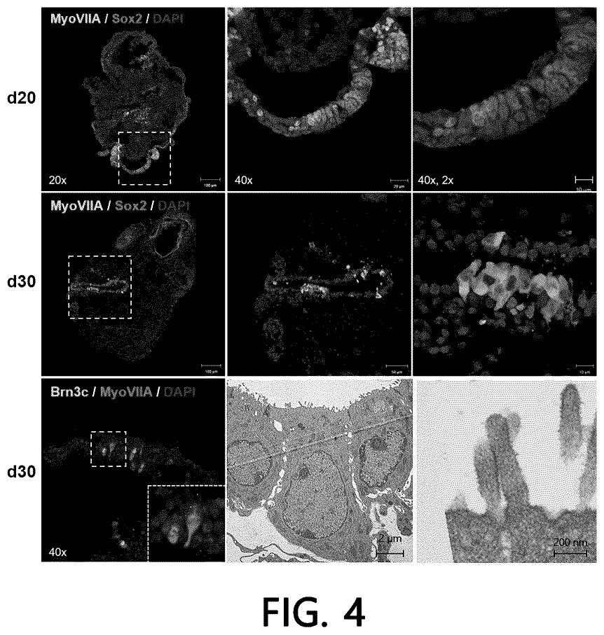

On the other hand, the organoid produced according to Example 1 formed a normal three-dimensional inner ear organoid structure (FIG. 3), and when the degree of sensory hair cell development was determined through Myosin VIIA, Brn3c and Sox2 immunostaining and the cilia was confirmed through transmission electron microscopy, it can be confirmed that it contained a greater number of hair cells than the DMSO control group (FIG. 4).

As shown in the above examples, the treatment of DAPT and CHIR-99021 during the production of inner ear organoids can affect the differentiation of hair cells, which can lead to different results depending on the treatment period, and it can be seen that DAPT and CHIR-99021 must be treated in the late stage of the organoid maturation step to be able to promote the differentiation of hair cells in the inner ear organoid.

The above description of the present invention is for illustrative purposes, and those skilled in the art will understand that it can be easily modified into other specific forms without changing the technical spirit or essential features of the present invention. Therefore, the exemplary embodiments described above should be understood as illustrative in all respects and not restrictive.

1. A method for producing an inner ear organoid having improved hair cell differentiation ability, comprising the steps of:
a) culturing embryonic stem cells or induced pluripotent stem cells to induce the formation of endoderm, ectoderm and otic vesicle of the cultured cells; and
(b) maturing by culturing the cells differentiated according to step (a) in organoid medium,
wherein a gamma secretase inhibitor and Wnt signaling activator are treated in step (b).

2. The method of claim 1, wherein the gamma secretase inhibitor and Wnt signaling activator are treated in the later stage of the maturing step.

3. The method of claim 1, wherein the gamma secretase inhibitor and Wnt signaling activator are treated on day 25 to day 35 after the start of differentiation when the stem cells in step (a) are embryonic stem cells, and are treated on day 80 to day 90 after the start of differentiation when the stem cells in step (a) are induced pluripotent stem cells.

4. The method of claim 1, wherein the gamma secretase inhibitor is DAPT (N—[N-(3,5-difluorophenacetyl)-1-alanyl]-S-phenylglycine t-butyl ester).

5. The method of claim 1, wherein the Wnt signaling activator is CHIR-99021.

6. The method of claim 1, wherein the Wnt signaling activator is treated at a concentration of 1 μM to 2.5 μM, and the gamma secretase inhibitor is treated at a concentration of 1 μM to 5 μM.

7. An inner ear organoid having improved hair cell differentiation ability, which is produced by the method of claim 1.
